(19) United States
(12) Patent Application Publication
(10) Pub. No.: US 2011/0152782 A1
(43) Pub. Date: Jun. 23, 2011

(54) Cover for the tip of a sharp medical device

(76) Inventor: Gary Christopher Jones, Lindfield (AU)

(21) Appl. No.: 12/989,401

(22) PCT Filed: Apr. 23, 2009
(86) PCT No.: PCT/AU2009/000507

§ 371 Date: Feb. 1, 2011

(30) Foreign Application Priority Data
Apr. 23, 2008 (AU) 2008902017

(51) Int. Cl. A61M 5/32

(52) U.S. Cl. 604/192

(57) ABSTRACT

A cover (40) to encompass the tip of a sharp medical device such as a needle (41). In particular the cover (40) is to cover the tip (42) of the needle (41). The cover (40) has a body (43) integrally formed of resilient material and having a passage (44) through which the needle (41) is to pass. The needle (41) resiliently deforms the body (43) so that the body (43) is urged into frictional engagement with the longitudinal outer surface (45) of the needle (41). The body (43) has a further passage (46) that intersects with the passage (44). An elongated flexible element (47) extends into the passage (46) and has an aperture (51) through which the needle (41) passes. Upon a user gripping the element (47) and moving the element (47) into the direction (52), the body (43) passes along the needle (41) until the tip (42) enters the body (43) and passes the passage (46). Upon the needle (41) passing the passage (46) the element (47) is released thereby leaving the body (43) covering the tip (42) with the passage (44) diminishing in transverse cross section to inhibit movement of the body (43) back along the needle (41). The body (43) is retained in position by being resiliently deformed and urged into contact with the surface (44).

### TECHNICAL FIELD

The present invention relates to a cover to encompass the tip of a sharp medical device, typically the tip may be the tip of a needle of a syringe, catheter or cannula or other sharp medical device such as a trocar, scalpel or drill. More particularly, but not exclusively, the present invention relates to a cover to encompass the tip of a syringe needle.

### BACKGROUND OF THE INVENTION

Medical devices such as needles, catheters, cannulae, trocars, scalpels and drills typically have sharp tips that facilitate penetration of anatomical structures such as the skin.

A conventional medical needle consists of a solid shaft, with a sharp tip at one end and an opening at the other end, whereby a structure may be pierced and a flexible thread drawn therethrough.

A conventional medical catheter consists of a hollow tube for insertion into a body cavity, duct or vessel, thereby allowing drainage or injection of fluids or access by surgical instruments. Typically a catheter is a rigid tube, such as a hollow hypodermic needle, which terminates with a sharp tip. A catheter may also be a flexible tube such as a cannula, which may be introduced into the body using a trocar or similar penetrating device that terminates with a sharp tip.

A conventional medical scalpel consists of a handle to which a flat blade is attached, such blade typically having one or more sharp edges and a sharp tip.

A conventional medical drill consists of a cylindrical shaft, typically with sharp edges on its outer surface, which terminates with a sharp tip.

These conventional devices are used in enormous numbers.

A disadvantage of the abovementioned conventional devices is that the tip is often exposed and can result in injuries. Where such injuries occur after clinical use, the tip is contaminated whereupon the risk to an injured person of infection by blood-borne pathogens is significantly increased. This can result in negative and significant (potentially fatal) health outcomes for an injured person. More particularly, there are enormous numbers of “needle stick” injuries following the use of hypodermic syringes to which a sharp hollow needle is attached.

The above disadvantage is exacerbated by the cost associated with the care and management of injured persons, as well as occupational health and safety management issues, and litigation.

Various systems have been proposed to cover the tips of sharp medical devices, particularly the tips of hollow needles such as those used in hypodermic syringes. These previous systems are often complex thereby adding to their unreliability and costs. These factors have inhibited their wide acceptance.

Described in U.S. Pat. Nos. 4,929,241, 4,955,866, 5,053,017, 5,344,480, 5,662,610 and 6,406,459 as well as USA Patent Publication 2004/0049155 are devices that are mounted on the needle of a syringe and are movable therealong so as to cover the needle tip when the syringe is no longer to be used. Several of the devices described in these documents suffer a common disadvantage in that a user could inadvertently move the cover beyond the needle tip so that the cover is no longer mounted thereon, thereby exposing the needle tip. A further disadvantage is that the cover must be gripped and manually moved along the needle, whereby the user could inadvertently touch the needle shaft or the needle tip. Accordingly the use of these above mentioned covers may actually increase the risk of incurring a needle-stick injury and thereby exacerbate the extent of the problems associated with needle stick injuries.

### OBJECT OF THE INVENTION

It is the object of the present invention to overcome or substantially ameliorate at least one of the above disadvantages.

### SUMMARY OF THE INVENTION

There is disclosed herein a cover for a tip of a medical device, the medical device having a body from which there extends an elongated part terminating at said tip, said cover including:

a cover body to be mounted on the elongated part for sliding movement therealong from a retracted position at which the cover body is to be spaced from the tip, to an extended position at which the tip is located within the cover body so as to be covered thereby; and

an elongated flexible element attached to the cover body and used by an operator so that upon tensioning the element said cover body is moved from the retracted position to the extended position, and wherein said flexible element is released from engagement with said cover body when said tip is located internally of said cover body.

Preferably, said cover body includes an engaging portion that is to be resiliently deformed so as to be urged into frictional engagement with the elongated part, with tensioning of said elongated flexible element deforming said engaging portion to reduce or overcome frictional engagement with the elongated part.

Preferably, upon release of said elongated flexible element said engaging portion increases in frictional engagement with said elongated part.

Preferably, said cover body has a pair of aligned apertures through which the elongated part is to pass, and said engaging portion has an aperture through which the elongated part passes.

Preferably, said cover includes a catch operatively associated with said cover body and said elongated flexible element to retain said element attached to said cover body until said tip enters said cover body at which said catch releases said elongated flexible element.

Preferably, said elongated flexible element has an aperture through which said elongated part passes so that upon said tip passing through the aperture of said flexible element, said flexible element is released from said cover body.

Preferably, said cover body is moved angularly relative to said elongated part at said extended position to locate said tip in said cover body.

Preferably, said body is formed from resilient material that has a passage through which the elongated part is to extend so as to resiliently deform the body so that the body is urged into frictional engagement with the elongated part.

Preferably, said passage is a first passage, and said body has a second passage intersecting the first passage with said element extending through said second passage so as to be engageable with the elongated part, with the element being released from the elongated part when the tip thereof passes the second passage.

Preferably, said element has an aperture aligned with said first passage.

Preferably, said body is integrally formed of said resilient material.

Preferably, said body is hollow and has a forward aperture and a rear aperture with the apertures being aligned so that the elongated part may pass through the body, and a retaining assembly mounted internally of the body releasably securing the element to the body so that upon movement of the elongated part passed a predetermined position locating the tip internally of the body, the element is released and the retaining assembly engages the elongated part to inhibit movement of the body relative to the elongated part.

Preferably, said assembly includes a sleeve through which the elongated part is to pass, and a clutch member fixed to the body and engaged by the sleeve, the clutch member being urged into engagement with the elongated part to inhibit movement of the body relative to the elongated part, with said sleeve being engaged by the element to move the sleeve relative to the body to move the clutch to a position permitting movement of the elongated part relative to the body.

Preferably, the cover further includes at least one pawl to engage the elongated part to at least substantially inhibit movement of the tip from within the body.

Preferably, said body is hollow and said cover includes a clutch member located internally of said body, said clutch member being attached to said body and urged to engage the elongated part to at least substantially inhibit movement of the elongated part relative to the body, and wherein said element is engaged with said clutch member to provide for movement of the elongated part relative to the body with the element being released from the clutch member when said elongated part moves beyond a predetermined position locating said tip internally of said body.

Preferably, the cover further includes at least one pawl to prevent movement of the tip from internally of the body.

Preferably, said body is hollow and has a forward aperture and a rear aperture through which the elongated part is to pass, and said cover includes a clutch member located internally of the body and to engage the elongated part, the clutch member being configured to be resiliently deformed by the elongated part to provide for movement of the elongated part relative to the body, with said clutch member being operative to engage the elongated part to at least inhibit movement of the elongated part relative to the body when said tip enters said body so as to move beyond a predetermined position.

Preferably, said elongated flexible element is a thread or string or ribbon or strip having a tag.

Preferably, said tag includes a layer of absorbent or adhesive material.

### BRIEF DESCRIPTION OF THE DRAWINGS

Preferred forms of the present invention will now be described by way of example with reference to the accompanying drawings wherein:

FIG. 1 is a series of schematic side elevations of a hypodermic syringe and needle tip cover therefor;

FIG. 2 is a series of schematic sectioned side elevations of a modification of the syringe and needle tip cover of FIG. 1;

FIG. 3 is a series of schematic sectioned side elevations of a modification of the tip cover of FIG. 1;

FIG. 4 is a series of schematic sectioned side elevations of a modification of the needle tip cover of FIG. 1;

FIG. 5 is a series of schematic sectioned side elevations of a modification of the needle tip cover of FIG. 1;

FIG. 6 is a series of schematic sectioned side elevations of a modification of the needle tip cover of FIG. 1;

FIG. 7 is a series of schematic sectioned side elevations of a modification of the needle tip cover of FIG. 1;

FIG. 8 is a series of schematic sectioned side elevations of a still further modification of the needle tip cover of FIG. 1;

FIG. 9 is a series of schematic sectioned side elevations of a further needle tip cover.

FIG. 10 is a series of schematic sectioned side elevations of a further needle tip cover;

FIG. 11 is a series of schematic sectioned side elevations of a further needle tip cover;

FIG. 12 is a series of schematic sectioned side elevations of a further needle tip cover;

FIG. 13 is a series of schematic sectioned side elevations of a further needle tip cover;

FIG. 14 is a series of schematic sectioned side elevations of a further needle tip cover;

FIG. 15 is a series of schematic sectioned side elevations of a still further needle tip cover;

FIG. 16 is a series of schematic elevations of an elongated flexible element;

FIG. 17 is a series of schematic elevations of a modification of the elongated flexible element of FIG. 16;

FIG. 18 is a series of schematic elevations of a modification of the elongated flexible element of FIG. 17.

### DETAILED DESCRIPTION OF THE PREFERRED EMBODIMENTS

As mentioned previously, the present invention relates to a cover to encompass the tip of a sharp medical device. In the following embodiments the cover is described by way of a preferred construction, that is, a cover used with the needle of a syringe. However, it should be appreciated that the cover could also be employed with other sharp medical devices that have an elongated part such as a scalpel blade or drill.

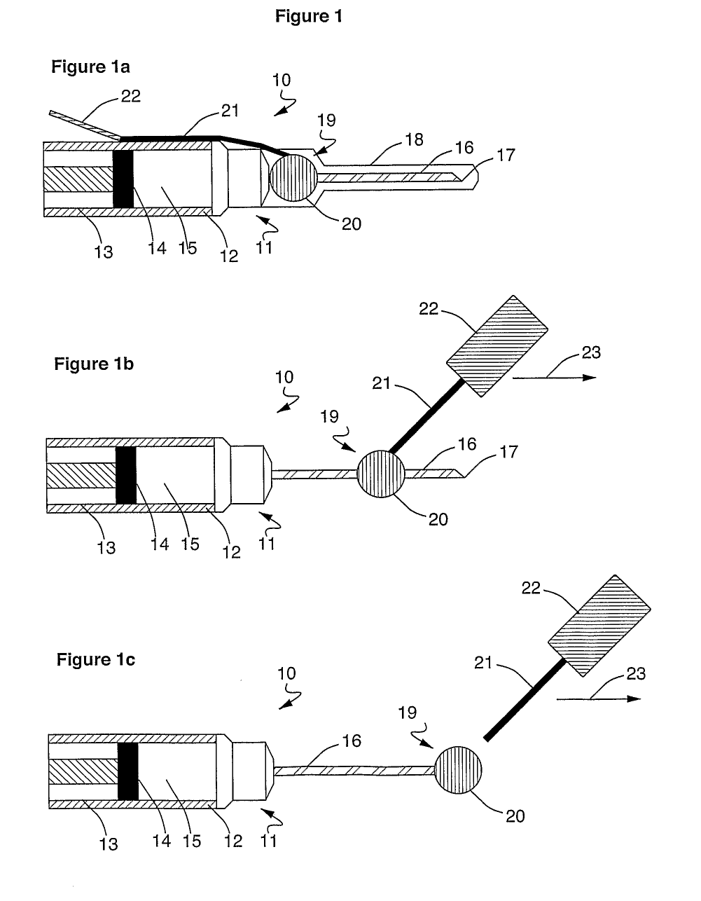

FIG. 1 is a series of schematic part-sectioned side elevations of the operation of a hypodermic syringe and a needle tip cover therefor. This combination includes a hypodermic syringe 10 including a syringe body 11 having a cylinder 12 with a longitudinally extending inner cylindrical surface 13 that slidably receives a plunger 14.

The cylinder 13 and plunger 14 co-operate to provide a variable volume chamber 15 that receives a fluid to be injected or withdrawn, with reduction in the volume of the chamber 15 resulting in the fluid being injected or withdrawn via the needle 16. The needle 16 extends longitudinally from the cylinder 12 and has a terminal end fixed thereto and extends therefrom to a sharp needle tip 17. Initially the hypodermic syringe 10 includes a cap 18 enclosing the needle 16. The cap 18 is removed when the syringe 10 is to be used.

Operatively associated with the syringe 10 is a cover 19 that includes a cover body 20 slidably mounted on the needle 16 so as to be movable therealong from a retracted position to an extended position. In the retracted position the body 20 is located spaced from the tip 17 and is preferably located adjacent the cylinder 12. In the extended position the body 20 encloses the tip 17 so that the tip 17 is no longer exposed. The retracted position is shown in FIG. 1a. In FIG. 1b the cover 19 is illustrated in transit being slidably moved along the needle 16 from the retracted position to the extended portion, while in FIG. 1c the cover 20 encloses the tip 17.

Attached to the body 20 are elongated flexible elements 21 that may be one or more tapes, ribbons or lengths of thread. Preferably, the elements 21 have a terminal end fixed to the body 20 and extend therefrom and are fixed to a tab 22 that may be releasably fixed to the cylinder 12. Typically a release adhesive would secure the tab 22 to the cylinder 12 so that a user may grip a raised portion of the tab 22 and apply a force thereto in the direction 23. Movement of the tab 22 in the direction 23 and tensioning of the elements 21 moves the body 20 from the retracted position to the extended position. At the extended position, the body 20 engages the needle 16 adjacent the tip 17 so that the elements 21 release the body 20 due to the continued application of tension to the elements 21.

For example, the elements 21 may be secured to the body 20 by a releasable adhesive or may be at least partly embedded therein so that upon tension in the elements 21 exceeding a predetermined tension, the elements 21 release the body 20.

In one embodiment to cause the elements 21 to disengage from the body 20, the degree of engagement of the body 20 with the needle 16 adjacent the tip 17 needs to be increased.

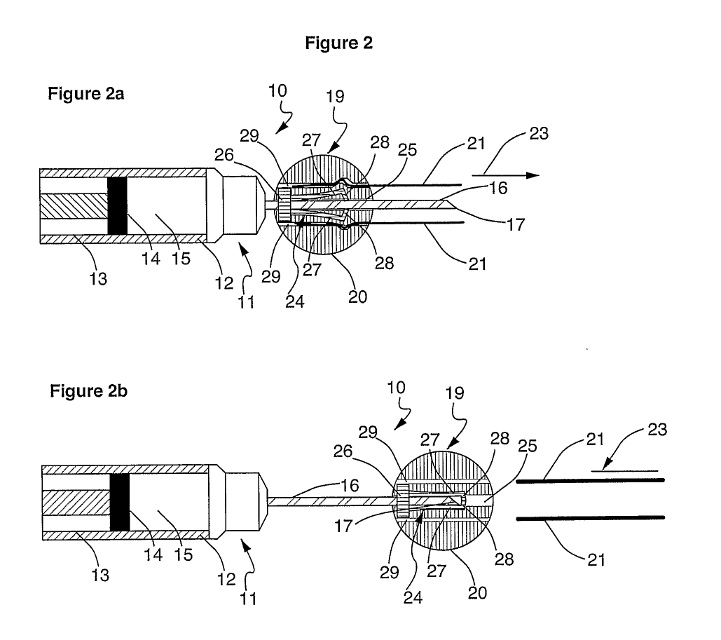

In the embodiment of FIG. 2 this is achieved by a resilient lever device 24 embedded in the body 20. The body 20 includes a through passage 25 via which the needle 16 passes. Located around and engaged with the needle 16 is a device base 26 through which the needle 16 also passes. The base 26 is slidable along the needle 16. Formed integral with and extending longitudinally forward from the base 26 are resilient levers 27 having stop arms 28 resiliently urged into engagement with the needle 16 by resilient deformation of the levers 27. The stop arms 28 have limited surface contact with the longitudinal exterior surface of the needle 16 and therefore provide for sliding movement therealong. When the elements 21 are tensioned and pulled in the direction 23, the body 20 with the device 24 embedded therein is moved to the extended position. At the extended position the stop arms 28 move past the tip 17 with the result that the arms 27 move into engagement with the longitudinal outer peripheral surface of the needle 16 increasing frictional engagement therewith. This increase in frictional engagement results in the elongated flexible elements 21 disengaging from the body 20. The levers 27 therefore ensure that the body 20 is retained at the extended position with the tip 17 embedded therein. Preferably the device 24 is integrally moulded from plastics material.

Preferably the elements 21 extend into passages 29 in the body 20. As an example end extremities of the elements 21 can be located internally of the body 20 during moulding of the body 20. Additionally a portion of the elongated flexible element 21 can be engaged by the stop arms 28 when the stop arms 28 are engaged with the longitudinal peripheral surface of the needle 16 to aid in retaining the elements 21 engaged with the body 20. Upon the stop arms 28 passing the tip 17, the stop arms 28 deflect inwardly releasing the elements 21.

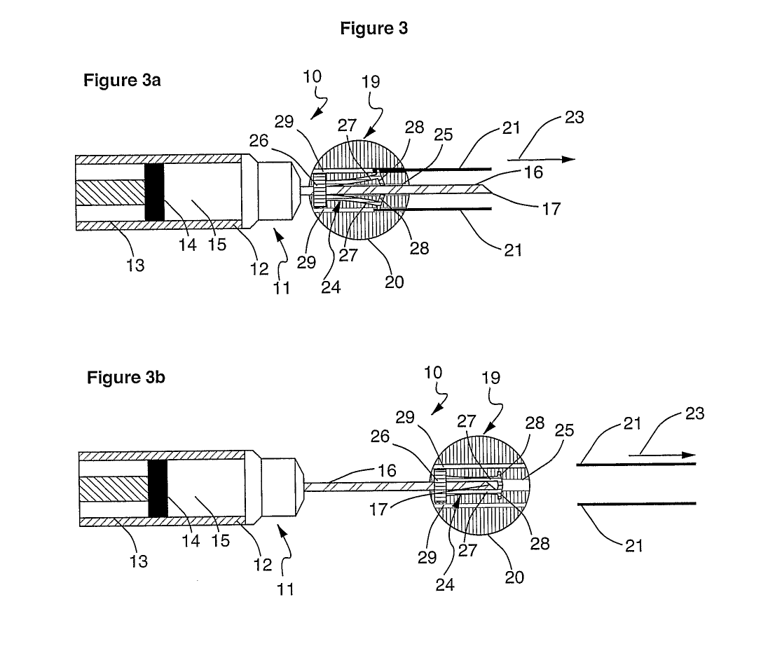

In the embodiment of FIG. 3, the end extremities of the elements 21 are provided with apertures within which portion of the stop arms 28 is located so that upon movement of the stop arms 28 inwardly when they pass the tip 17, the elements 21 are released from the device 24 and therefore the body 20.

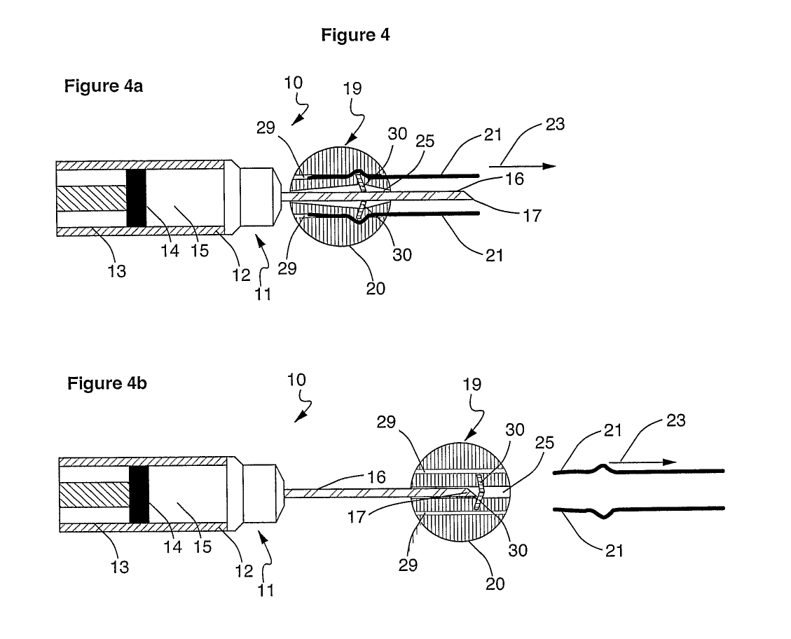

In the embodiment of FIG. 4, moulded in the body 20 are element engaging members 30 that are urged outwardly to engage the elements 21 to retain them attached to the body 20. The members 30 by being moulded within the body 20 are resiliently urged into engagement with the longitudinal peripheral surface of the needle 16 and slide therealong until they pass the tip 17. Thereafter they move inwardly so as to cover the tip 17 and release the elements 21.

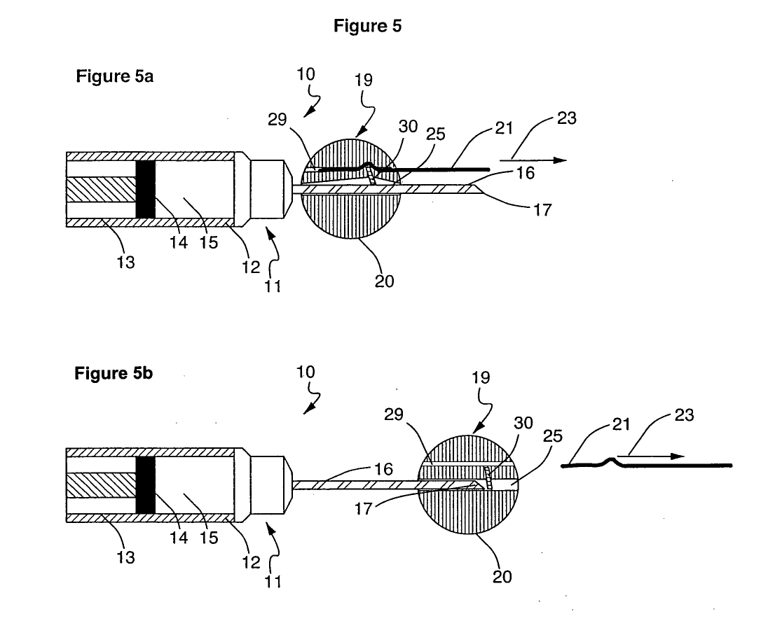

In the embodiment of FIG. 5, the body 20 has a single member 30, the member 30 again releasing the element 21 upon the member 30 passing the tip 17. The member 30 would be typically moulded within the body 20 and resiliently urged into contact with the longitudinal outer peripheral surface of the needle 16.

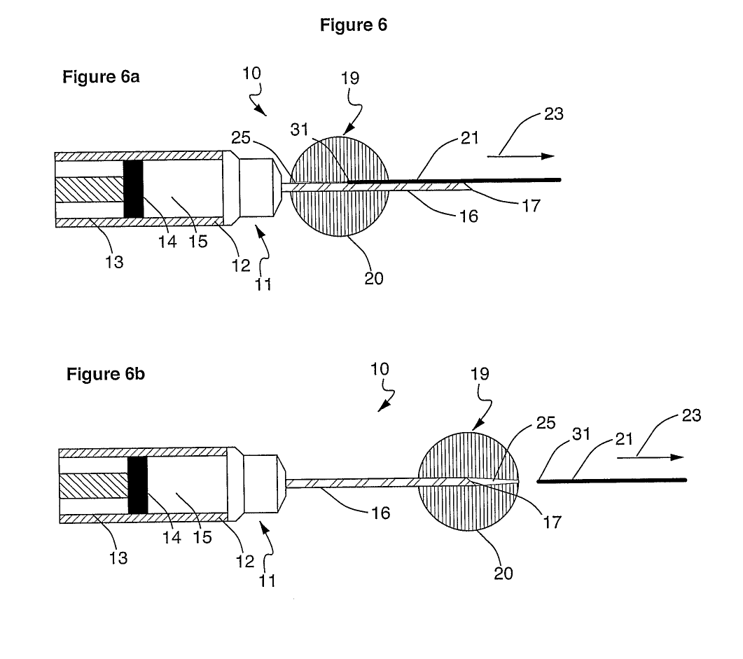

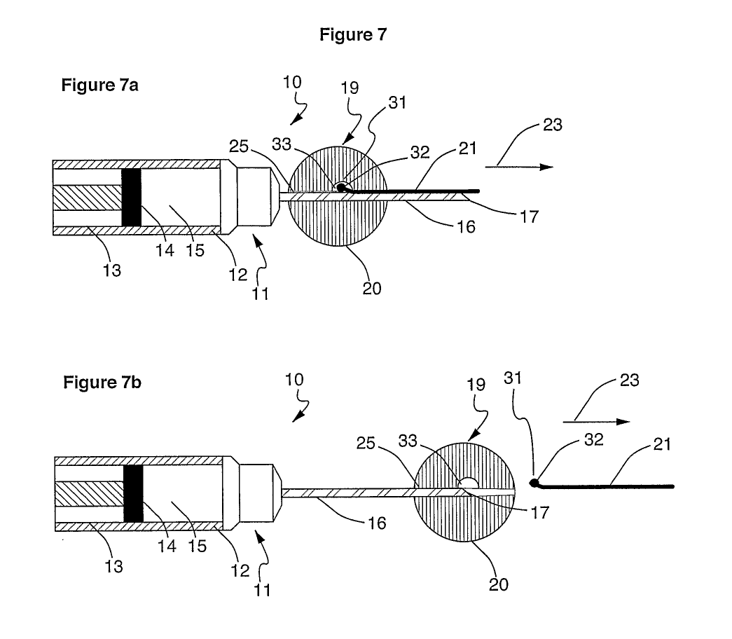

In the embodiments of FIGS. 6 and 7, the body 20 is resiliently deformed and urged into contact with the outer peripheral surface of the needle 16. Sandwiched between the body 20 and the needle 16 is the element 21. Upon the end extremity 31 of the element 21 passing the tip 17, the element 21 is released thereby leaving the body 20 covering the tip 17. The frictional engagement of the body 20 with the outer peripheral surface of the needle 16 retains the body 20 in position covering the tip 17.

In the embodiment of FIG. 7, the end extremity 31 is provided with an enlarged portion 32 projecting into a cavity 33 of the body 20. Upon the enlarged portion 32 passing the tip 17, the element 21 is released. Again the body 20 is resiliently urged into frictional engagement with the outer peripheral surface of the needle 16 so that the body 20 upon being released from the element 21 is retained covering the tip 17.

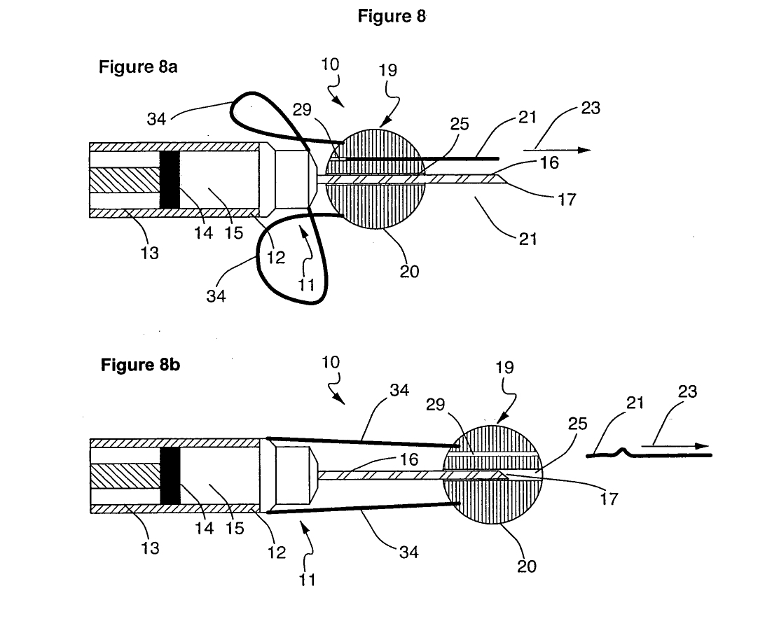

In the embodiment of FIG. 8, again the body 20 is resiliently urged into frictional engagement with the outer peripheral surface of the needle 16. The element 21 is secured so as to extend inwardly of the body 20. Upon the body 20 reaching and covering the tip 17, elongated anchoring members (flexible elongated members) 34 limit movement of the body 20 away from the cylinder 12 so that the body 20 is positioned covering the tip 17. Upon this position being reached the element 21 separates from the body 20.

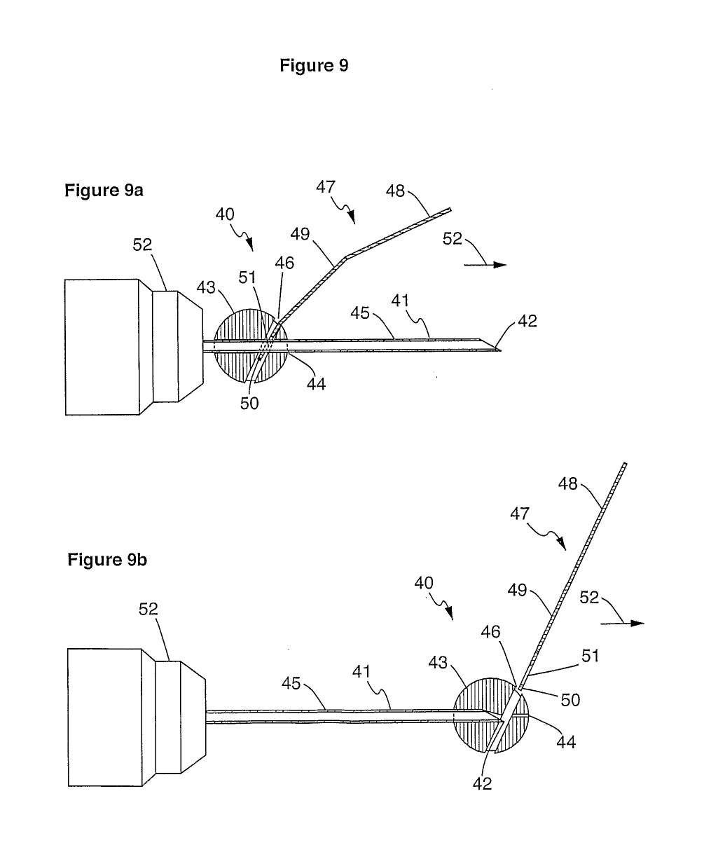

In the embodiment of FIG. 9 there is provided a needle tip cover 40. The cover 40 is operatively associated with a needle 41 having a tip 42. The cover 40 includes a body 43 formed of resilient material. As particular examples the body 43 may be formed of synthetic or natural rubber, or of any one of a number of plastics materials including foamed plastics material. The body 43 has a passage 44 through which the needle 41 passes. Preferably the body 43 is integrally formed, and moulded so as to incorporate the passage 44.

When the body 43 is located on the needle 41 the body 43 is resiliently deformed so as to be urged into frictional engagement with the outer longitudinally extending surface 45 of the needle 41.

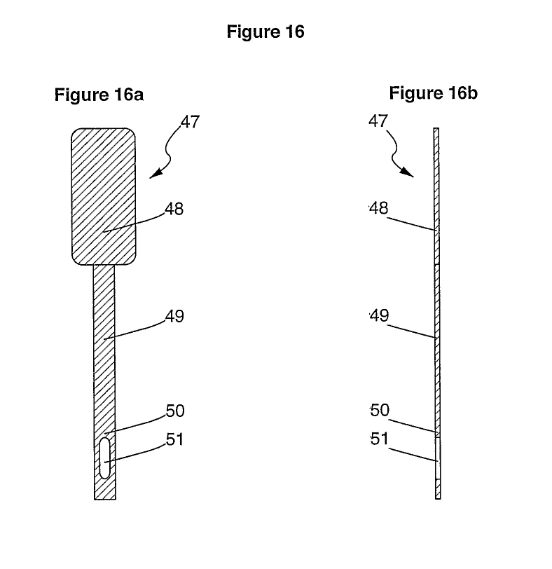

The body 43 includes a further passage 46 that intersects with the passage 45. Initially located in the passage 46 is an elongated flexible element 47 as depicted in FIG. 16.

In the inoperative (retracted) position, the body 43 is located adjacent the syringe body 52, that is a position spaced from the tip 42. The end portion 50 is located in the passage 46 with the aperture 51 aligned with the passage 44 so the needle 41 passes through the aperture 51 and the aperture 44. The stem 49 extends from within the passage 46.

In operation of the above described cover 40 a user grips the tab 48 and pulls the tab 48 in the direction 52, thereby moving the body 43 along the surface 45 from the retracted position (FIG. 9a) to the extended position (FIG. 9b). In the extended position the body 43 covers the tip 42, with that portion of the body 43 that extends beyond the tip 42 moving inwardly so as to reduce the diameter of the passage 44 extending beyond the tip 42. This prevents, or at least substantially inhibits, movement of the body 43 in the reverse direction of the direction 52 whereby the tip 42 would otherwise be exposed. The body 43 is retained in the extended position by frictional engagement with the surface 45 of needle 41.

When the passage 46 moves beyond the tip 41 the end portion 50 is released from the needle 41 thereby releasing the flexible element 47.

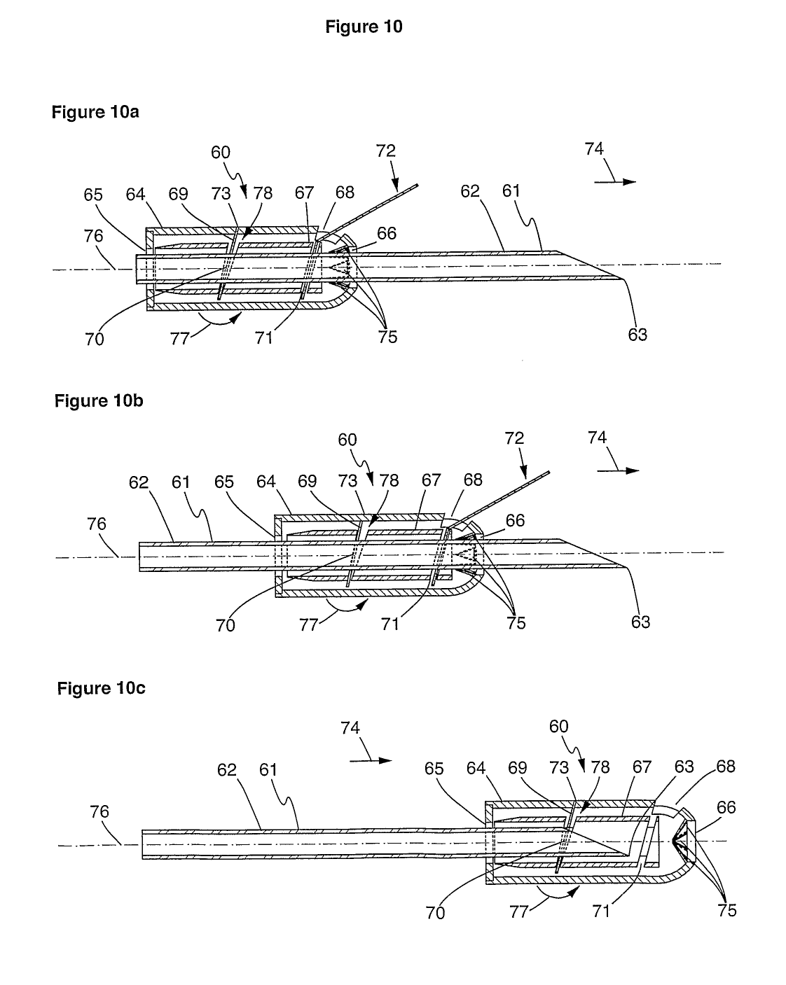

In the embodiment of FIG. 10 there is provided a needle tip cover 60. In this embodiment the cover 60 is operatively associated with a needle 61 having a longitudinally outer surface 62 and a tip 63.

The cover 60 has an outer hollow body 64 having a rear aperture 65 and a forward aperture 66 that are aligned so that the needle 61 can pass through the cover 60. Located internally of the outer body 64 is a retaining assembly 78 that includes a sleeve 67 and a clutch member 69. Sleeve 67 surrounds the needle 61 but has an internal diameter greater than the external diameter of the needle 61. A slot 68 extends transversely through the body of sleeve 67 and receives the clutch member 69. The clutch member 69 is preferably in the form of a plate or strip with a central aperture 70 through which the needle 61 passes. A forward portion of the sleeve 67 is provided with a passage 71 in which there is located an elongated flexible element 72. The element 72 is of a similar construction to the element 47 described in FIG. 16, so as to have a stem 49 and end portion 50 with an aperture 51. The aperture 51 is aligned with the apertures 65 and 66 so the needle also passes through the aperture 50.

The clutch member 69 is relatively stiff and is preferably formed from a strip of resilient material such as plastic or metal. The clutch member 69 has an end portion 73 embedded in the outer body 64 so as to be urged to an inclined position as shown in the drawings. In this regard it should be appreciated the diameter of aperture 70 is only slightly larger than the diameter of needle 61 so that when the clutch member 69 is in the inclined position as discussed the clutch member 69 securely engages the outer surface 62 of the needle 61.

When the element 72 is gripped by a user and pulled in the direction of the arrow 74 the sleeve 67 moves in the direction of the arrow 64 causing angular movement of the clutch member 69 in the direction of the arrow 77 about the end portion 73. This releases the clutch member 69 from secure engagement with the surface 62 thereby permitting movement of the cover 60 in the direction 74 (as shown in FIG. 10b). As the passage 71 passes the tip 63 (as shown in FIG. 10c) the elongated member 72 is released by passing beyond the tip 63. The sleeve 67 is then also released from the element 72 thereby permitting the clutch member 69 to move to its natural position, angularly in the opposite direction to the direction 77, thereby again securely engaging the surface 62 and preventing, or at least substantially inhibiting, movement of the cover 60 in the direction 74. In that regard if a user grips the outer body 64 and attempts to move the outer body 64 in the direction 74, the clutch member 69 will increase in engagement with the surface 62 therefore making it even more difficult to move the cover 60 in direction 74.

Preferably the outer body 64 is provided with a plurality of fingers 75 adjacent the aperture 66 and extending rearwardly therefrom (opposite the direction 74). The fingers 74 are resiliently urged against the surface 62 so that upon the tip 73 passing rearwardly of the fingers 75, the fingers 75 move radially inward relative to the longitudinal axis 76 of the needle 61 thereby preventing or at least substantially inhibiting movement of the cover 60 rearwardly (a direction opposite the direction 74). This thereby inhibits any subsequent exposure of the tip 63.

FIG. 10a illustrates the cover 60 before use with the element 72 untensioned, that is with the cover 60 in a rest position exposing the tip 63. FIG. 10b illustrates the cover 60 during use, with the element 72 tensioned so as to provide for movement of the cover 60 in the direction 74. FIG. 10c illustrates the cover 60 after use, in the position covering the tip 63.

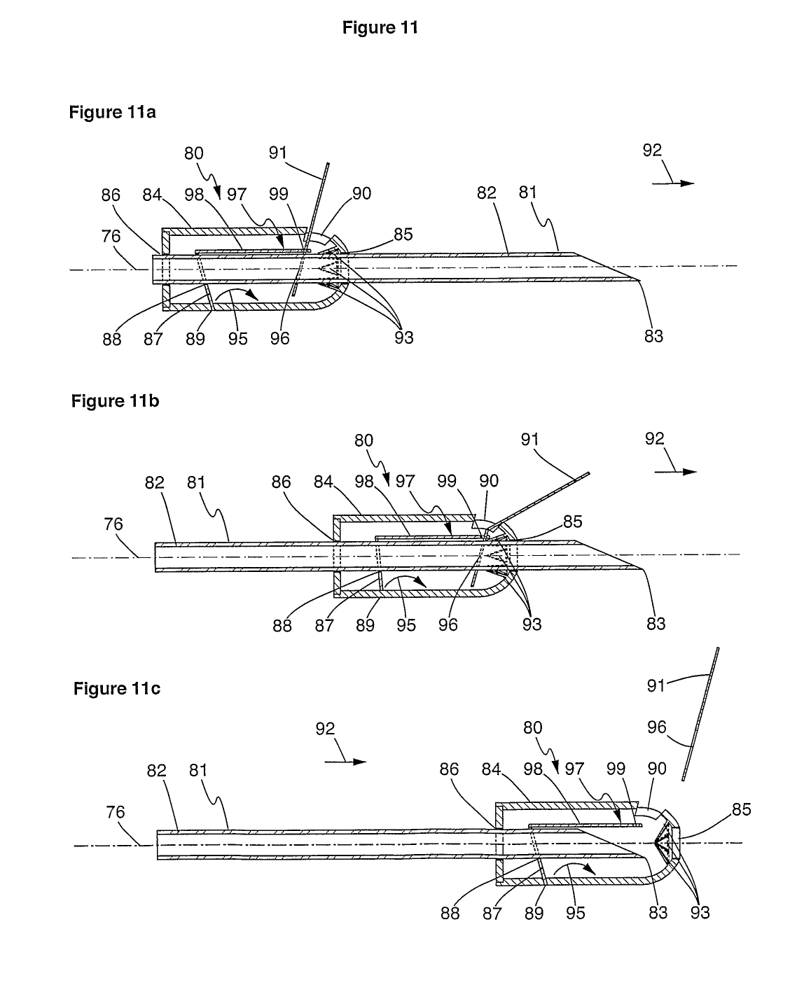

In the embodiment of FIG. 11 there is provided a needle tip cover 80 mounted on a needle 81 having a longitudinal outer surface 82 and a tip 83. The cover 80 has a hollow outer body 84 having a forward aperture 85 and a rear aperture 86, with the apertures 85 and 86 aligned so that needle 81 can pass therethrough.

Extending through a passage 90 of the outer body 84 is an elongated flexible element 91 that is substantially the same construction as the element 47 of FIG. 16. In this embodiment the element 91 has an aperture 96 through which the needle 81 passes.

Located internally of the body 84 is a clutch member 97 having a longitudinal portion 98 extending longitudinally relative to the needle 81, and a transverse portion 87. The transverse portion 87 has an aperture 88 that is slightly larger than the diameter of the needle 81 and through which the needle 81 passes. The transverse portion 87 has an end portion 89 embedded in the outer body 84 so that transverse portion 87 is resiliently urged into engagement with the surface 82 so as to retain the cover 80 secured to the needle 81.

The longitudinal portion 98 has an opening 99 that is slightly larger than the width of flexible element 91, and through which the element 91 freely passes. When a user grips the element 91 and pulls the element 91 in the direction 92, the portion 98 of clutch member 97 is moved in the direction of the direction 92, thereby causing angular movement of the transverse portion 87 in the direction 95 about the end portion 89. This releases the engagement of the transverse portion 87 with respect to the surface 82 thereby providing for movement of the cover 80 in the direction 92.

When the elongated flexible element 91 passes beyond the tip 83 (as shown in FIG. 11c) the element 91 is released. This in-turn releases the clutch member 97 so that the transverse portion 87 moves angularly in a direction opposite the direction 95 relative to the end portion 89 so that again the transverse portion 87 engages the surface 82 to prevent further movement of the cover 80 in the direction 92.

If an attempt is made to move the cover 80 in the direction 92 by directly gripping the outer cover 84, the portion 87 even more securely engages the surface 82, thereby further resisting the motion.

Preferably the outer cover 84 has a plurality of resilient fingers 93 adjacent to aperture 85 urged into contact with the surface 82. The fingers 83 project rearwardly (a direction opposite the direction 92) from the aperture 85, and deflect radially inward relative to the longitudinal axis 94 when the opening 85 projects sufficiently beyond the tip 83. The fingers 93 when moved radially inward, engage the tip 83 upon an attempt to move the cover 80 rearward (a direction opposite the direction 92). This thereby inhibits any subsequent exposure of the tip 83.

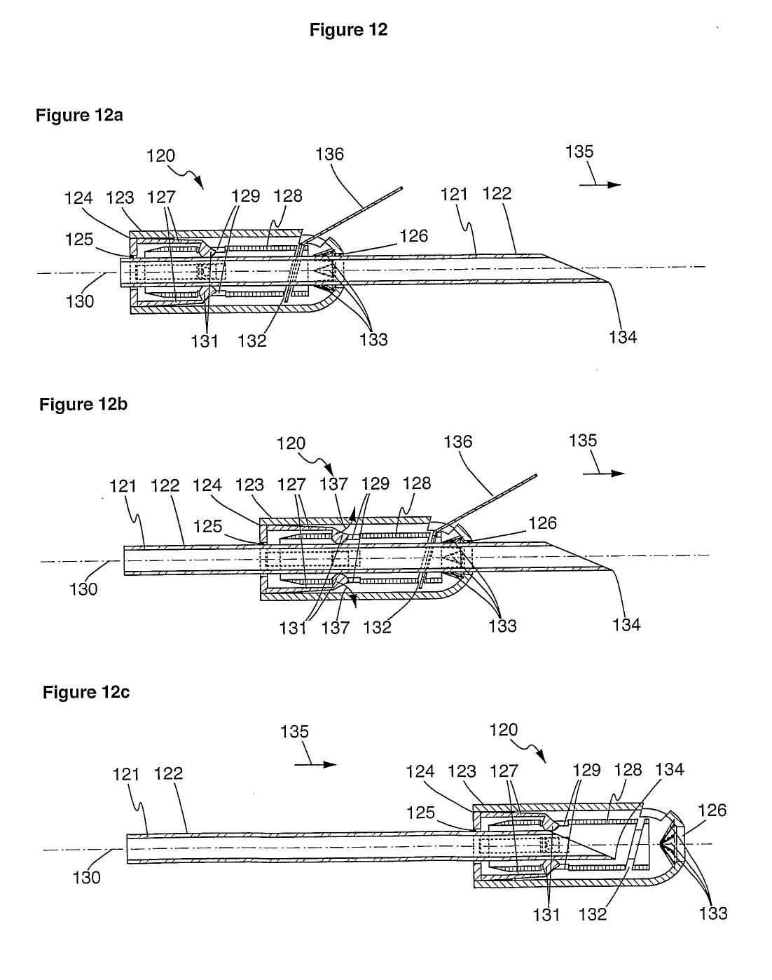

In the embodiment of FIG. 12 there is provided a cover 120 for a needle 121 having a longitudinal outer surface 122 and a tip 134. The cover 120 has a hollow outer body 123 having a rear wall 124 with a central aperture 125, and a forward opening 126 that is aligned with the opening 125 so that the needle 121 can pass through the body 123.

Located internally of the body 123 is a plurality of pawls 127. The pawls 127 may be formed separately to the body 123 or may be integrally formed with the end wall 124. The pawls 127 are resiliently urged into contact with the surface 122.

Also located internally of the body 123 is sleeve 128 that surrounds the needle 121 but has an internal diameter greater than the external diameter of the needle. The sleeve 128 has a plurality of apertures 129 arranged angularly about the longitudinal axis 130. The apertures 129 are aligned with end parts 131 of the pawls 127 so the end parts 131 are allowed to engage the surface 122.

The sleeve 128 has a transverse passage 132 that receives an elongated flexible element 136 of substantially the same construction as the element 47 of FIG. 16.

Preferably, the body 123 adjacent the aperture 126 has a plurality of resilient fingers 133 that are urged to moved radially inwardly relative to the axis 130 as described in the previous embodiments.

FIG. 12a shows the cover 120 in the retracted position. After the needle is used, the cover 120 is moved along the needle 121 by pulling the flexible element 136 in the direction 135, whereupon the sleeve 128 slides forward, causing pawls 131 to move outwards in direction 137, thereby reducing friction between 131 and 122 sufficiently to allow 120 to slide along 122 in the direction 135, as shown in FIG. 12b.

When the flexible element 136 moves past the tip 134, the flexible element 136 is released, whereby the pawls return to their natural position, leaving the cover 120 in the extended position encompassing the tip 134, as shown in FIG. 12c.

When the cover 120 projects sufficiently beyond the tip 134, the fingers 133 move radially inward preventing rearward movement of the cover 120 to again expose the tip 134. However forward movement (movement in the direction of the arrow 135) is prevented or at least substantially inhibited by end parts 131 being urged into even further firm contact with the surface 122.

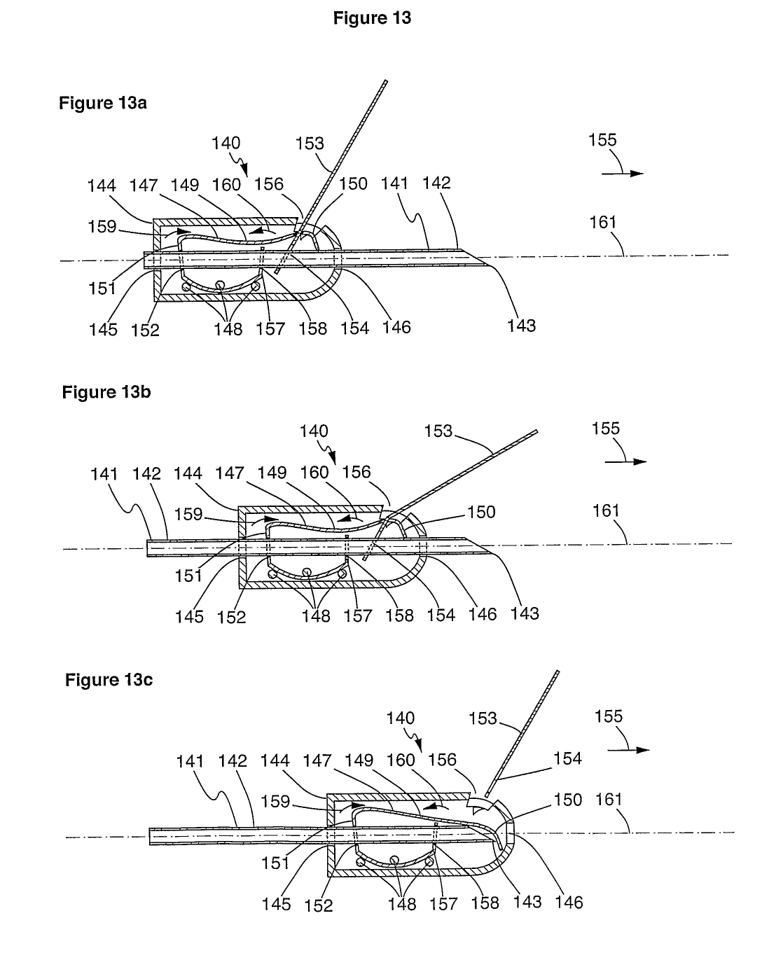

In the embodiment of FIG. 13 there is schematically depicted a cover 140 for a needle 141. The needle 141 has a longitudinal peripheral surface 142 and a tip 143. The cover 140 includes an outer hollow body 144 with a rear aperture 145 and a forward aperture 146, with the apertures 145 and 146 aligned so that the needle 141 may pass therethrough.

Located internally of the outer body 144 is a clutch member 147, preferably formed from a strip of resilient material such as plastic or metal. More particularly the body 144 has several transverse pins 148 that mount the clutch member 147 on the body 144.

The clutch member 147 has a longitudinal part 149 having a forward end 150 slidable on, and resiliently urged to contact with, the surface 142. Extending generally transverse relative to the needle 141 are rear part 151 and forward part 157 of the clutch member 147.

The rear part 151 has an aperture 152, and the forward part 157 has an aperture 158, each aperture being aligned with the apertures 145 and 146 so that the needle 141 can pass therethrough. The apertures 152 and 158 are only slightly larger than the diameter of the needle 141, so that when parts 151 and 157 are in an inclined position with respect to axis 161 of needle 141, they securely engage the outer surface 142 of the needle 141.

The body 144 has a passage 156 with an elongated flexible element 153 passing therethrough. The element 153 is of substantially the same construction as the element 47 of FIG. 16 in that it has an aperture 154 through which the needle 141 passes.

In use of the above described cover 140, a user grips the flexible element 153 and pulls the element 153 in the direction 155. The longitudinal part 149 of clutch member 147 is moved in the direction 155, thereby causing angular movement of the transverse portion 151 in the direction 159. This releases the engagement of the transverse portion 151 with respect to the surface 141, thereby providing for movement of the cover 140 in the direction 155. At the same time, forward motion of the cover 140 causes angular movement of the transverse portion 157 in the direction 160. This releases the engagement of the transverse portion 157 with respect to the surface 141.

This causes the cover 140 to move forward as shown in FIG. 13b until the forward aperture 146 of the body 144 and forward end 150 of part 147 pass beyond the tip 143 as shown in FIG. 13c. Forward end 150 of clutch member 147 then moves to a position between the tip 143 and the aperture 146, whereby the tip 143 is prevented from again passing through the aperture 146.

Further pulling of the flexible element 153 in the direction 155 causes the element 153 to pass beyond the tip 143, whereby the element 153 is released. The clutch member 147 then returns to its natural shape, causing the parts 151 and 157 to more securely engage the surface 142 to prevent, or at least substantially inhibit, movement of the cover 140 along the needle 141.

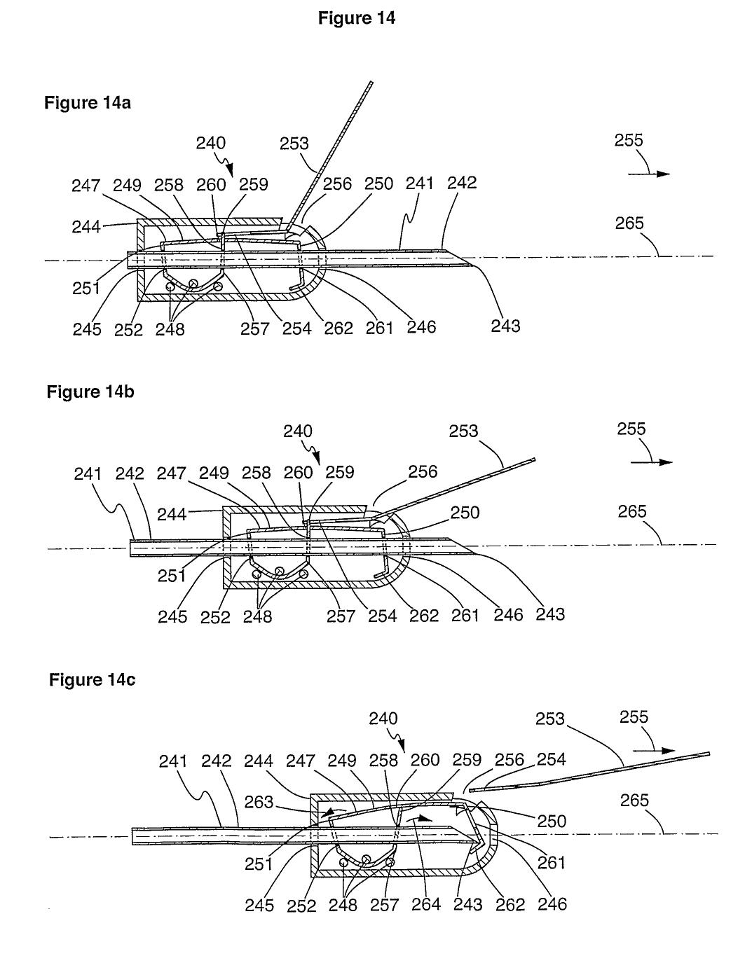

In the embodiment of FIG. 14 there is schematically depicted a cover 240 for a needle 241. The needle 241 has a longitudinal peripheral surface 242 and a tip 243. The cover 240 includes an outer hollow body 244 with a rear aperture 245 and a forward aperture 246, with the apertures 245 and 246 aligned so that the needle 241 may pass therethrough.

Located internally of the outer body 244 is a clutch member 247, preferably formed from a strip of resilient material such as plastic or metal. More particularly the body 244 has several transverse pins 248 that mount the clutch member 247 on the body 244.

The clutch member 247 has a longitudinal part 249 having a forward end 250 and an end part 262. Extending generally transverse relative to the needle 241 are rear part 251 and middle part 257 of the clutch member 247. The middle part 257 has a protuberance 259 and longitudinal part 249 has an aperture 260.

The rear part 251 has an aperture 252, the middle part 257 has an aperture 258 and the forward part 250 has an aperture 261. The apertures 252 and 258 are only slightly larger than the diameter of the needle 241, so that when parts 251 and 257 are in an inclined position, they securely engage the outer surface 242 of the needle 241.

During assembly, protuberance 259 is inserted through aperture 260 in longitudinal part 249, whereby end parts 251 and 257 are held in a position relatively perpendicular to the axis 265, and apertures 252 and 258 are aligned with apertures 245 and 246. Subsequently, end part 250 is moved towards axis 265 so as to bring aperture 261 into alignment with the apertures 245 and 246 so that the needle 241 can pass therethrough. In the perpendicular positions, parts 251 and 257 are relatively free to move along the outer surface 242 of the needle 241. Aperture 261 is urged into frictional contact with surface 242 of needle 241, whereby cover 240 is loosely retained on needle 242.

The body 244 has a passage 256 with an elongated flexible element 253 passing therethrough. The element 253 is of substantially the same construction as the element 47 of FIG. 16, in that it has an aperture 254 through which, in this embodiment, protuberance 259 passes.

In use of the above described cover 240, a user grips the flexible element 253 and pulls the element 253 in the direction 255. This causes flexible element 253 to pull on protuberance 259, whereby the cover 240 moves along needle 241 in direction 255 as shown in FIG. 14b. When forward aperture 261 of body 247 passes beyond tip 243 as shown in FIG. 14c, forward part 250 of clutch member 247 is released from engagement with the needle 241, whereby the longitudinal part 249 of clutch member 247 returns to its natural position, moving in a direction away from the needle. End part 262 then moves to a position between the tip 243 and aperture 246, whereby the tip 243 is prevented from again passing through the aperture 246.

At the same time, protuberance 259 is released from engagement in aperture 260, allowing transverse portions 251 and 257 to return to their natural positions. Angular movement of portion 251 in the direction 263, together with angular movement of portion 257 in the direction 264, causes the parts 251 and 257 to more securely engage the surface 242 to prevent, or at least substantially inhibit, movement of the cover 240 along the needle 241.

At the same time, protuberance 259 is withdrawn from opening 254 in flexible element 253, whereupon element 253 is released.

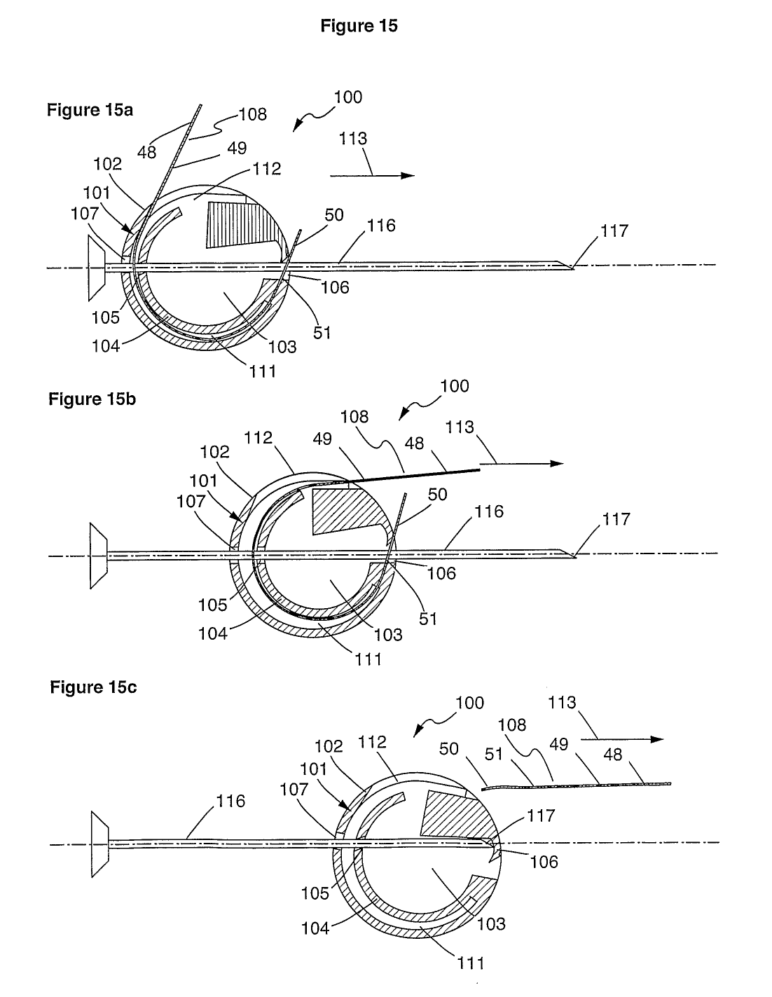

In the embodiment of FIG. 15 there is schematically depicted a cover 100. In this embodiment the cover 100 has a hollow body 101 provided by a spherical wall 102. The wall 102 encompasses a cavity 103. Integrally formed with the wall 102 is a needle engagement portion 104 having an aperture 105. The body 102 has apertures 106 and 107. The apertures 105, 106 and 107 are alignable so that the needle 116 may pass therethrough.

The cover 100 also includes an elongated flexible element 108 of a construction similar to that of the element 47 described in FIG. 16, so as to have a tag 48, a stem 49 and end portion 50 with an aperture 51.

The portion 104 is generally arcuate so as to be generally parallel to but spaced from the wall 102 by means of a slot 111.

The ribbon 108 is inserted in the slot 111 and exits therefrom so that the aperture 51 is aligned with the apertures 105, 106 and 107.

During assembly an operator via opening 112 inserts the ribbon 108 and resiliently deforms the portion 104 so that the apertures 105, 106 and 107 together with the aperture 51 are aligned thereby providing for insertion of the needle 116 until it projects entirely through the cover 100 as seen in FIG. 15a. Accordingly, if the tag 48 is pulled in the direction 113, the ribbon 108 is tensioned until the body 102 moves longitudinally along the needle 116. Eventually the needle tip 117 is located in the cavity 103 at which time the needle tip 117 becomes withdrawn through the aperture 51 thereby releasing the ribbon 108. The ribbon 108 therefore detaches from the body 102 leaving the body 102 encompassing the needle tip 117. Preferably the wall 102 has a socket 118 within which the tip 117 is located.

Once the needle tip passes entirely through the aperture 106, the body 101 moves angularly in the direction 114 about the aperture 107. That is an axis transverse of the needle at the aperture 107.

In the several of the embodiments, the needle engagement portions are resiliently urged into contact with the needle to aid in retaining the respective cover positioned on the needle adjacent the needle body, that is in the retracted position. When the elongated flexible element is tensioned, the associated needle engaging portion is moved to reduce or overcome frictional engagement between the cover and the needle, thereby providing for easier movement of the cover body along the needle. When the cover body encompasses the needle tip, the elongated flexible element is released, the needle engaging portion remains in, or is released to again be urged into, frictional engagement with the needle so as to retain the body encompassing the needle tip.

In FIG. 16 is illustrated a flexible element 47 may be a tag or other flexible element and includes a tab 48 that is enlarged so as to be gripped by a user. A stem 49 is fixed to the tab 48 and extends from the tab 48 so as to terminate with an end portion 50. The end portion 50 is provided with an aperture 51 large enough for a needle to pass therethrough.

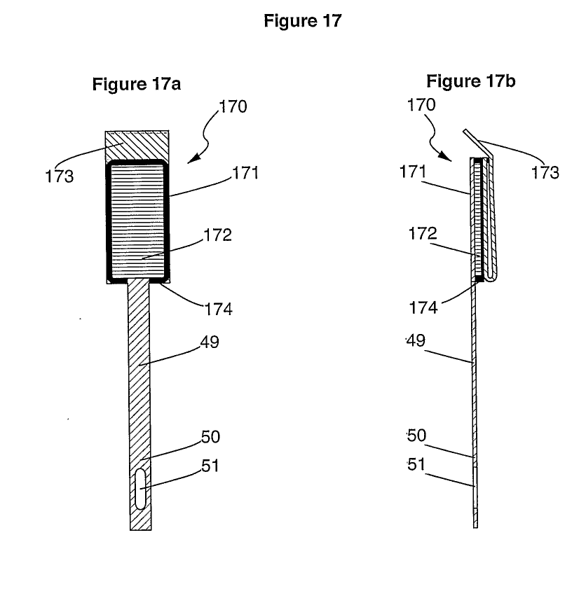

In FIG. 17 is illustrated another example of the elongated flexible element 170 including a tag 171. The tag 171 can also perform further useful tasks during extraction of the needle. For example, the tag 171 could have a layer 172 of absorbent material that could be placed over the wound to assist in withdrawal of the needle and to absorb body fluids. To maintain it in a relative hygienic state, a strip 173 may be also part of a protective layer that overlays the absorbent layer 172 and is held in place by a layer of adhesive 174.

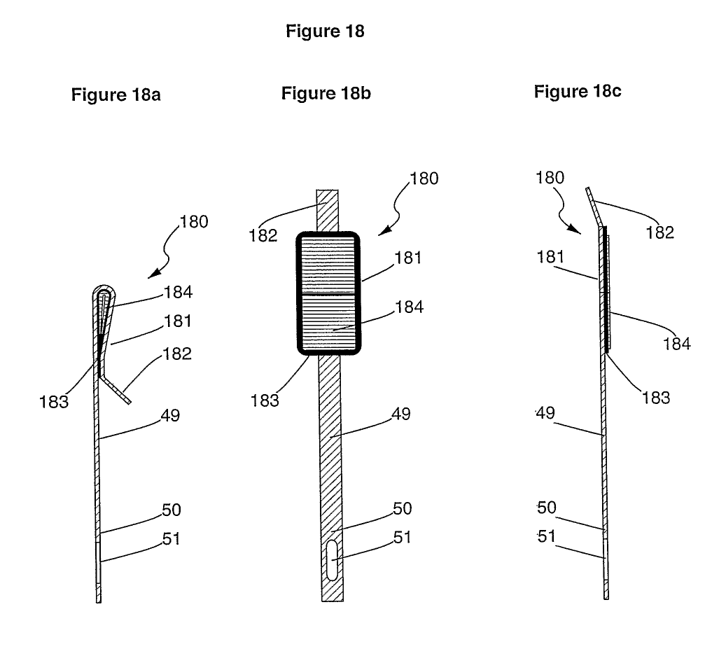

In FIG. 18 is illustrated another example of the elongated flexible element 180 and tag 181, incorporating a layer of absorbent material 184. To maintain it in a relative hygienic state, the tag 181 could be folded as shown in FIG. 18a, and provided with a strip 182 that would be gripped to aid in unfolding the tag 181. Preferably the strip 182 would have a releasable adhesive layer 183 so that it could be engaged with the opposing side of the tag 181 when folded. The adhesive layer 183 could be arranged whereby the absorbent layer 184 remains affixed to the patient after use.

A particular advantage of each of the above described preferred embodiments is that each needle tip cover, when each located so as to encompass a needle tip, releases the elongated flexible element so that the cover is located in the desired position covering the needle tip, that is the release inhibits the cover being pulled off the needle. Another advantage of at least some of the embodiments is that frictional contact between the cover and the needle at the extended positions of the cover is increased.

1. A cover for a tip of a medical device, the medical device having a body from which there extends an elongated part terminating at said tip, said cover including:
a cover body to be mounted on the elongated part for sliding movement therealong from a retracted position at which the cover body is to be spaced from the tip, to an extended position at which the tip is located within the cover body so as to be covered thereby; and
an elongated flexible element attached to the cover body and used by an operator so that upon tensioning the element said cover body is moved from the retracted position to the extended position, and wherein said flexible element is released from engagement with said cover body when said tip is located internally of said cover body.

2. The cover of claim 1 wherein said cover body includes an engaging portion that is to be resiliently deformed so as to be urged into frictional engagement with the elongated part, with tensioning of said elongated flexible element deforming said engaging portion to reduce or overcome frictional engagement with the elongated part.

3. The cover of claim 2 wherein upon release of said elongated flexible element said engaging portion increases in frictional engagement with said elongated part.

4. The cover of claim 3 wherein said cover body has a pair of aligned apertures through which the elongated part is to pass, and said engaging portion has an aperture through which the elongated part passes.

5. The cover of claim 1 wherein said cover includes a catch operatively associated with said cover body and said elongated flexible element to retain said element attached to said cover body until said tip enters said cover body at which said catch releases said elongated flexible element.

6. The cover of claim 1 wherein said elongated flexible element has an aperture through which said elongated part passes so that upon said tip passing through the aperture of said flexible element, said flexible element is released from said cover body.

7. The cover of claim 6 wherein said cover body is moved angularly relative to said elongated part at said extended position to locate said tip in said cover body.

8. The cover of claim 1 wherein said body is formed from resilient material that has a passage through which the elongated part is to extend so as to resiliently deform the body so that the body is urged into frictional engagement with the elongated part.

9. The cover of claim 8 wherein said passage is a first passage, and said body has a second passage intersecting the first passage with said element extending through said second passage so as to be engageable with the elongated part, with the element being released from the elongated part when the tip thereof passes the second passage.

10. The cover of claim 9 wherein said element has an aperture aligned with said first passage.

11. The cover of claim 9, wherein said body is integrally formed of said resilient material.

12. The cover of claim 1 wherein said body is hollow and has a forward aperture and a rear aperture with the apertures being aligned so that the elongated part may pass through the body, and a retaining assembly mounted internally of the body releasably securing the element to the body so that upon movement of the elongated part past a predetermined position locating the tip internally of the body, the element is released and the retaining assembly engages the elongated part to inhibit movement of the body relative to the elongated part.

13. The cover of claim 12 wherein said assembly includes a sleeve through which the elongated part is to pass, and a clutch member fixed to the body and engaged by the sleeve, the clutch member being urged into engagement with the elongated part to inhibit movement of the body relative to the elongated part, with said sleeve being engaged by the element to move the sleeve relative to the body to move the clutch to a position permitting movement of the elongated part relative to the body.

14. The cover of claim 13 further including at least one pawl to engage the elongated part to at least substantially inhibit movement of the tip from within the body.

15. The cover of claim 1 wherein said body is hollow and said cover includes a clutch member located internally of said body, said clutch member being attached to said body and urged to engage the elongated part to at least substantially inhibit movement of the elongated part relative to the body, and wherein said element is engaged with said clutch member to provide for movement of the elongated part relative to the body with the element being released from the clutch member when said elongated part moves beyond a predetermined position locating said tip internally of said body.

16. The cover of claim 15 further including at least one pawl to prevent movement of the tip from internally of the body.

17. The cover of claim 1 wherein said body is hollow and has a forward aperture and a rear aperture through which the elongated part is to pass, and said cover includes a clutch member located internally of the body and to engage the elongated part, the clutch member being configured to be resiliently deformed by the elongated part to provide for movement of the elongated part relative to the body, with said clutch member being operative to engage the elongated part to at least inhibit movement of the elongated part relative to the body when said tip enters said body so as to move beyond a predetermined position.

18. The cover of claim 1 wherein said elongated flexible element is a thread or string or ribbon or strip having a tag.

19. The cover of claim 18 wherein said tag includes a layer of absorbent or adhesive material.

20. (canceled)
